(19) United States
(12) Patent Application Publication
(10) Pub. No.: US 2026/0233154 A1
(43) Pub. Date: Aug. 13, 2026

(54) GAS TREATMENT METHOD AND GAS TREATMENT DEVICE

(71) Applicants: KABUSHIKI KAISHA KOBE SEIKO SHO (KOBE STEEL, LTD.), Hyogo (JP); National University Corporation Tokai National Higher Education and Research System, Nagoya-shi, Aichi (JP)

(72) Inventors: Akira KISHIMOTO, Kobe-shi, Hyogo (JP); Norihide MAEDA, Kobe-shi, Hyogo (JP); Mai YOSHIZAWA, Kobe-shi, Hyogo (JP); Hiroshi MACHIDA, Aichi (JP); Koyo NORINAGA, Aichi (JP)

(73) Assignees: KABUSHIKI KAISHA KOBE SEIKO SHO (KOBE STEEL, LTD.), Hyogo (JP); National University Corporation Tokai National Higher Education and Research System, Nagoya-shi, Aichi (JP)

(21) Appl. No.: 19/156,283

(22) PCT Filed: Dec. 28, 2023
(86) PCT No.: PCT/JP2023/047315

§ 371 Date: Aug. 13, 2025

(30) Foreign Application Priority Data
Feb. 14, 2023 (JP) 2023-020773

(51) Int. Cl. B01D 53/14; B01D 53/79; C07C 1/12; C07C 9/04; C07C 29/151; C07C 31/04; C07C 31/08

(52) U.S. Cl. CPC B01D 53/1475; B01D 53/1418; B01D 53/1425; B01D 53/79; C07C 1/12; C07C 9/04; C07C 29/1518; C07C 31/04; C07C 31/08; B01D 2251/202; B01D 2257/504

(57) ABSTRACT

A gas treatment device includes an absorber that brings a gas to be treated containing carbon dioxide in contact with a treatment liquid to cause the carbon dioxide contained in the gas to be treated to be absorbed by the treatment liquid, a regenerator that heats the treatment liquid that has absorbed the carbon dioxide to separate the carbon dioxide from the treatment liquid, a reactor that uses the gas separated from the treatment liquid in the regenerator and containing the carbon dioxide to synthesize methane, and an introduction path that introduces, into the regenerator, a separation promoting gas that promotes separation of the carbon dioxide from the treatment liquid. The introduction path includes a heat transfer path that preheats the separation promoting gas before introduction into the regenerator by using heat generated in the reactor as a heat source.

### TECHNICAL FIELD

The present invention relates to a gas treatment method and a gas treatment device.

### BACKGROUND ART

Conventionally, there has been known a gas separation method of separating an acidic compound contained in a gas to be treated by bringing the acidic compound in contact with a treatment liquid. In this type of gas separation method, the gas to be treated is brought in contact with the treatment liquid in the absorber, the acidic compound in the gas to be treated is absorbed by the treatment liquid, and the treatment liquid is heated in the regenerator to separate the acidic compound from the treatment liquid. On the other hand, Patent Literature 1 below discloses a system provided with a methanation reactor in addition to a CO2 separator. In this system, heat generated in the methanation reactor is used for regeneration of the treatment liquid in a regeneration unit in the CO2 separator. Patent Literature 2 below also discloses that reaction heat generated in a methane production facility is supplied to a reboiler in a CO2 separation and recovery device.

Non-Patent Literature 1 discloses a treatment liquid that is liquid-phase separated into a first phase part (for example, an amine phase) having a high content ratio of an acidic compound and a second phase part (for example, an ether phase) having a low content ratio of the acidic compound. This literature describes that the use of such a treatment liquid makes it possible to reduce the regeneration energy of the treatment liquid. Non-Patent Literature 2 below discloses that hydrogen gas is blown when an acidic compound is desorbed from an absorption liquid and a treatment liquid is regenerated. In this literature, it is described that the regeneration of the absorption liquid can be promoted and the regeneration temperature can be lowered by blowing hydrogen gas.

As disclosed in Patent Literatures 1 and 2, when heat generated in the methanation reactor is used for regeneration of the treatment liquid in the CO2 separator, energy required for regeneration of the treatment liquid can be reduced. As disclosed in Non-Patent Literature 2, it is also possible to reduce the energy required for regeneration of the treatment liquid by interposing hydrogen during regeneration of the treatment liquid. Therefore, if not only the heat generated in the methanation reactor is used in the regeneration step of the treatment liquid, but also the hydrogen gas is introduced into the regenerator in which the regeneration step is performed, it can be expected to further reduce the regenerated energy. However, even if hydrogen gas is introduced into the regenerator, heat from the methanation reactor is consumed for heating the hydrogen gas. Therefore, even if heat generated in the methanation reactor is introduced into the regenerator, the heat cannot be effectively used for regeneration of the treatment liquid.

### CITATION LIST

### Patent Literature

Patent Literature 1: JP 2015-51901 A
Patent Literature 2: JP 2020-63206 A

### Non-Patent Literature

Non-Patent Literature 1: Hiroshi Machida et al., “Development of phase separation solvent for CO2 capture by aqueous (amine+ether) solution”, Journal of Chemical Thermodynamics, (U.S.), Elsevier Ltd., 2017, Vol. 113, p. 64-70
Non-Patent Literature 2: Hiroshi Machida et al., “Energy-Saving CO2 Capture by H2 Gas Stripping for Integrating CO2 Separation and Conversion Processes” ACS Sustainable Chem. Eng. 2020, 8, 23, 8732-8740

### SUMMARY OF INVENTION

An object of the present invention is to effectively reduce energy required for regeneration of a treatment liquid.

A gas treatment method according to an aspect of the present invention includes an absorption step of bringing a gas to be treated containing an acidic compound that generates an acid when dissolved in water in contact with a treatment liquid in an absorber to cause the acidic compound contained in the gas to be treated to be absorbed by the treatment liquid, a regeneration step of heating the treatment liquid that has absorbed the acidic compound in a regenerator to separate the acidic compound from the treatment liquid, and a reaction step of causing a synthesis reaction by using the gas containing the acidic compound separated from the treatment liquid in the regeneration step. In the regeneration step, a separation promoting gas that promotes separation of the acidic compound from the treatment liquid is introduced into the regenerator, and the separation promoting gas is preheated by using heat generated in the reaction step as a heat source before being introduced into the regenerator.

A gas treatment device according to an aspect of the present invention includes an absorber that brings a gas to be treated containing an acidic compound that generates an acid when dissolved in water in contact with a treatment liquid to cause the acidic compound contained in the gas to be treated to be absorbed by the treatment liquid, a regenerator that heats the treatment liquid that has absorbed the acidic compound to separate the acidic compound from the treatment liquid, a reactor that causes a synthesis reaction by using a gas containing the acidic compound separated from the treatment liquid in the regenerator, and an introduction path that introduces, into the regenerator, a separation promoting gas that promotes separation of the acidic compound from the treatment liquid. The introduction path includes a heat transfer path that preheats the separation promoting gas before introduction into the regenerator by using heat generated in the reactor as a heat source.

### BRIEF DESCRIPTION OF DRAWINGS

FIG. 1 is a diagram schematically illustrating a gas treatment device according to a first embodiment.

FIG. 2 is a diagram schematically illustrating a gas treatment device according to a modification of the first embodiment.

FIG. 3 is a diagram schematically illustrating a gas treatment device according to a modification of the first embodiment.

FIG. 4 is a diagram schematically illustrating a gas treatment device according to a second embodiment.

FIG. 5 is a diagram schematically illustrating a gas treatment device according to a modification of the second embodiment.

FIG. 6 is a diagram schematically illustrating a gas treatment device according to a modification of the second embodiment.

FIG. 7 is a diagram schematically illustrating a gas treatment device according to a modification of the second embodiment.

FIG. 8 is a diagram schematically illustrating a gas treatment device according to a modification of the second embodiment.

### DESCRIPTION OF EMBODIMENTS

Embodiments will be described below with reference to the accompanying drawings. Note that the embodiments below are one example embodying the present invention, and are not intended to limit the technical scope of the present invention.

### First Embodiment

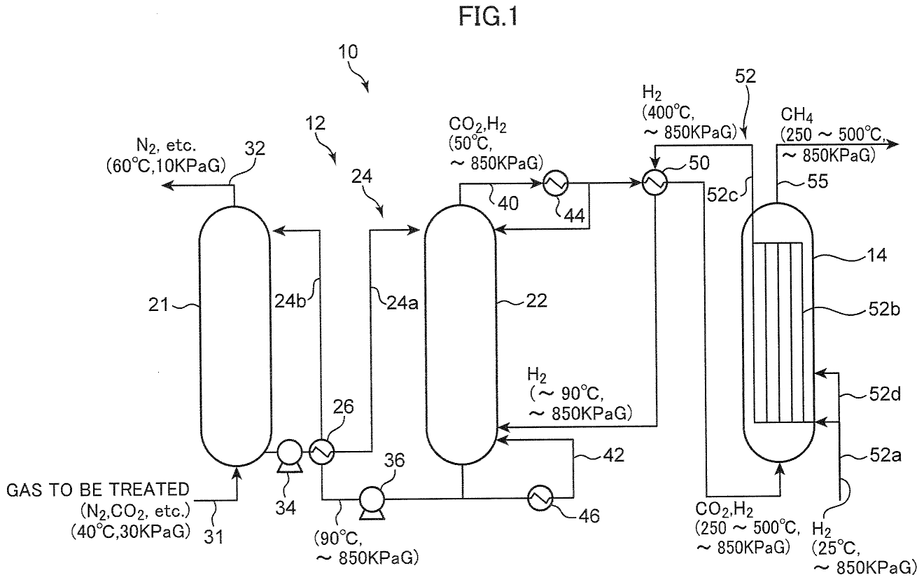

As illustrated in FIG. 1, a gas treatment device 10 according to a first embodiment includes a gas separator 12 used for separating an acidic compound from a gas to be treated containing the acidic compound using a treatment liquid, and a reactor 14 that causes a synthesis reaction by using the gas obtained by the gas separator 12. In the present embodiment, the acidic compound is carbon dioxide, and the reactor 14 is configured to synthesize methane, which is a hydrocarbon, by using a gas containing carbon dioxide separated by the gas separator 12. Therefore, the gas treatment device 10 also functions as a gas generator that generates hydrocarbon gas.

Since the gas treatment device 10 according to the present embodiment is configured as a device for producing a hydrocarbon gas, carbon dioxide as an acidic compound is to be treated. The acidic compound separated by the gas separator 12 is not limited as long as forming an acidic aqueous solution, and examples of the acidic compound include hydrogen chloride, carbon dioxide, sulfur dioxide, and carbon disulfide. Examples of the gas to be treated containing the acidic compound include industrial exhaust gas, process gas generated during purification, and natural gas.

The gas separator 12 includes an absorber 21, a regenerator 22, a circulation path 24, and a heat exchanger 26. The circulation path 24 includes a first flow path 24a that extracts the treatment liquid (rich liquid) from the absorber 21 and introduces the treatment liquid into the regenerator 22, and a second flow path 24b that extracts the treatment liquid (lean liquid) from the regenerator 22 and returns the treatment liquid to the absorber 21.

The heat exchanger 26 is connected to the first flow path 24a and the second flow path 24b, and causes heat exchange between the treatment liquid flowing through the first flow path 24a and the treatment liquid flowing through the second flow path 24b. Note that the heat exchanger 26 can be omitted.

An introduction path 31 for supplying the gas to be treated, a gas discharge path 32 for discharging gas after treatment, a first flow path 24a for sending the treatment liquid to the regenerator 22, and the second flow path 24b for returning the treatment liquid from the regenerator 22 to the absorber 21 are connected to the absorber 21. The introduction path 31 is connected to a lower portion of the absorber 21, and the gas discharge path 32 is connected to an upper portion of the absorber 21. The first flow path 24a is connected to the lower portion of the absorber 21. That is, the first flow path 24a is connected to a position where the treatment liquid accumulated in the absorber 21 can be extracted from the absorber 21. The second flow path 24b is connected to the upper portion of the absorber 21. That is, the second flow path 24b is connected to a position where the treatment liquid recirculated from the regenerators 22 can flow down from above.

By bringing the gas to be treated in contact with the treatment liquid, the absorber 21 causes the treatment liquid to absorb the acidic compound in the gas to be treated and discharges the gas from which the acidic compound has been removed. The absorber 21 is only required to bring the gas to be treated continuously in contact with the treatment liquid. The absorber 21 performs absorption of the acidic compound that is an exothermic reaction. When the acidic compound is carbon dioxide, the calorific value per 1 t of the absorption amount of the carbon dioxide is about 1.8 GJ. This reaction heat generated in the absorber 21 increases the temperatures of the gas to be treated and the treatment liquid.

The first flow path 24a and the second flow path 24b are connected to the regenerator 22. The first flow path 24a is connected to an upper portion of the regenerator 22, and introduces the treatment liquid absorbed the acidic compound and led out from the absorber 21 into the regenerator 22. The first flow path 24a is provided with a pump 34. The second flow path 24b is connected to a lower portion of the regenerator 22, and causes the treatment liquid stored in the regenerator 22 to be led out from the regenerator 22. The second flow path 24b is provided with a pump 36.

The regenerator 22 stores the treatment liquid that has absorbed the acidic compound, and heats the stored treatment liquid to desorb the acidic compound from the treatment liquid. The desorption of the acidic compound from the treatment liquid is an endothermic reaction. In the regenerator 22, when the treatment liquid is heated, not only the acidic compound is desorbed but also water in the treatment liquid is evaporated.

A heating flow path 42 and a supply path 40 are connected to the regenerator 22. The heating flow path 42 is a flow path for heating the treatment liquid in the regenerator 22, and is provided with a reboiler 46 that receives heat from a heat source. The heating flow path 42 causes the treatment liquid to flow out from the regenerator 22, heats the treatment liquid in the reboiler 46, and returns the treatment liquid to a bottom in the regenerator 22. The reboiler 46 may use recovered heat from the reactor 14 described later as a heat source, but may also be configured to directly or indirectly heat the treatment liquid by using an arbitrary heat source such as electricity, steam, or a burner, for example.

One end of the heating flow path 42 is connected to a lower part of the regenerator 22. The other end of the heating flow path 42 is connected to the second flow path 24b, but may be connected to the lower portion of the regenerator 22.

The supply path 40 supplies the reactor 14 with gas containing the acidic compound obtained in the regenerator 22. The supply path 40 is provided with a condenser 44 for cooling a mixed gas of a gas of the acidic compound evaporated from the treatment liquid and water vapor. When the mixed gas is cooled, the water vapor is condensed and thus can be separated. The separated water vapor is returned to the regenerator 22. The gas containing the acidic compound from which the water vapor has been separated is sent to the reactor 14. As the condenser 44, a heat exchanger using inexpensive cooling water such as river water can be used.

The reactor 14 causes a synthesis reaction by using a gas separated from the treatment liquid in the regenerator 22 and containing an acidic compound. That is, in the reactor 14, methane is synthesized by using a gas containing carbon dioxide and hydrogen gas supplied through an introduction path 52 described later. Specifically, in the reactor 14, a reverse shift reaction by carbon dioxide and hydrogen also proceeds to generate carbon monoxide and hydrogen.

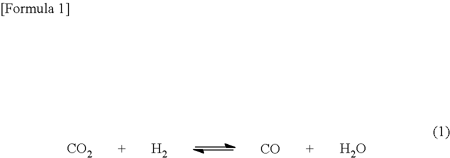

Since the equilibrium of the reaction according to this reaction formula (1) is biased to the right side (the side on which carbon monoxide is generated) as the temperature is higher, the reaction is performed at a higher temperature. In addition, methane is generated from carbon monoxide and hydrogen generated by the reverse shift reaction, and methane is also generated from carbon dioxide and hydrogen.

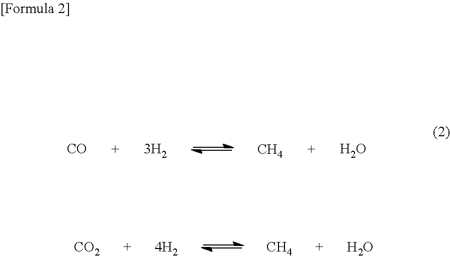

The reaction according to the reaction formula (2) is an exothermic reaction. Since this reaction is an equilibrium reaction, the product contains methane, hydrogen, and carbon dioxide.

The gas treatment device 10 is provided with the introduction path 52 for supplying the regenerator 22 with a gas for promoting separation of an acidic compound (hereinafter referred to as separation promoting gas). The introduction path 52 is a flow path through which the separation promoting gas flows, and a downstream end of the introduction path 52 is connected to the lower portion of the regenerator 22.

The separation promoting gas supplied to the regenerator 22 through the introduction path 52 is a gas that is hardly dissolved in the treatment liquid. That is, the separation promoting gas is hardly absorbed by the treatment liquid. Examples of the separation promoting gas include hydrogen gas, oxygen gas, and hydrocarbon gases such as methane gas. In the present embodiment, hydrogen gas is used as the separation promoting gas. Examples of the separation promoting gas include water vapor or a mixed gas of water vapor and a gas that is hardly dissolved in the treatment liquid. The gas partial pressure of the acidic compound in the regenerator 22 can be reduced by supplying the regenerator 22 with a gas that is hardly dissolved in the treatment liquid, water vapor, or a mixed gas of water vapor and a gas that is hardly dissolved in the treatment liquid. As a result, the separation of the acidic compound from the treatment liquid in the regenerator 22 can be promoted. Here, “hardly dissolved” in the treatment liquid may indicate that the solubility in the treatment liquid is a predetermined value or less. The separation promoting gas may be, for example, a gas that follows Henry's law and has a solubility of 1 mol or less in 100 g of the treatment liquid under the conditions of 0° C. and 100 kPa. The solubility of oxygen in water is 1.3×10′ mol/100 g, the solubility of methane in water is 8×10⁻⁴ mol/100 g, and the solubility of hydrogen in water is 9.5×10⁵ mol/100 g. On the other hand, since the solubility of ammonia in water is 6 mol/100 g, ammonia does not correspond to the separation promoting gas.

The introduction path 52 passes through the reactor 14. Therefore, the separation promoting gas flowing through the introduction path 52 receives heat (reaction heat) generated in the reactor 14, and introduces the heat into the regenerator 22. The introduction path 52 includes an upstream path 52a connected to a supply source of the separation promoting gas, a heat transfer path 52b connected to the upstream path 52a and positioned in the reactor 14, and a downstream path 52c connected to the heat transfer path 52b and connected to the regenerator 22. The supply source may be constituted by, for example, a water electrolyzer configured to be able to produce hydrogen by using renewable energy.

Since the heat transfer path 52b is disposed in the reactor 14, the separation promoting gas flowing through the introduction path 52 can receive heat (reaction heat) generated in the reactor 14 in the heat transfer path 52b. In other words, the separation promoting gas is preheated in the introduction path 52 by using the heat generated in the reactor 14 as a heat source before being introduced into the regenerator 22. The configuration of the heat transfer path 52b is not limited to a configuration in which the heat transfer path 52b passes through the reactor 14, and the heat transfer path 52b may be disposed in contact with a container constituting the reactor 14.

The introduction path 52 is provided with a branch path 52d that is connected to the upstream path 52a and introduces, into the reactor 14, a part of hydrogen gas which is the separation promoting gas flowing through the upstream path 52a. The configuration in which the hydrogen gas flows into the reactor 14 through the introduction path 52 is not limited, and the hydrogen gas may be supplied into the reactor 14 through a flow path separate from the introduction path 52. In this case, the branch path 52d is omitted.

A preheater 50 is disposed on the downstream path 52c of the introduction path 52. The preheater 50 is provided to preheat the gas introduced into the reactor 14 through the supply path 40 by the separation promoting gas heated (preheated) by receiving the heat generated in the reactor 14 before flowing into the reactor 14. The preheater 50 exchanges heat between the separation promoting gas flowing through the downstream path 52c in the introduction path 52 and the gas flowing through the supply path 40 to increase the temperature of the gas flowing through the supply path 40.

In the present embodiment, an absorbent capable of reversibly absorbing and desorbing an acidic compound is used as a treatment liquid (absorbent) used in the gas treatment device 10. The treatment liquid is, for example, an alkaline absorbent containing water, an amine compound, and an organic solvent. The amine compound may be 30 wt %, the organic solvent may be 60 wt %, and the water may be 10 wt %. The treatment liquid is preferably phase-separated by absorption of an acidic compound that is dissolved in water to generate an acid, but is not limited thereto. For example, the treatment liquid may be a treatment liquid prepared as an aqueous solution of an amine compound without using an organic solvent. The treatment liquid may be an amine compound, an organic solvent, an ionic liquid, a mixture thereof, an aqueous solution, or the like.

Examples of the amine compound include primary amines such as 2-aminoethanol (MEA: solubility parameter=14.3 (cal/cm³)^{1/2}) and 2-(2-aminoethoxy) ethanol (AEE: solubility parameter=12.7 (cal/cm³)^{1/2}), secondary amines such as 2-(methylamino) ethanol (MAE), 2-(ethylamino) ethanol (EAE) and 2-(butylamino) ethanol (BAE), and tertiary amines such as triethanolamine (TEA), N-methyldiethanolamine (MDEA), tetramethylethylenediamine (TEMED), pentamethyldiethylenetriamine (PMDETA), hexamethyltriethylenetetramine and bis (2-dimethylaminoethyl) ether.

Examples of the organic solvent include 1-butanol (solubility parameter=11.3 (cal/cm³)^{1/2}), 1-pentanol (solubility parameter=11.0 (cal/cm³)^{1/2}), octanol, diethylene glycol diethyl ether (DEGDEE), and diethylene glycol dimethyl ether (DEGDME), and a plurality of the above may be mixed and used.

In a case where the solubility parameter of each of the amine compound and the organic solvent falls within a predetermined range, the treatment liquid is two-phase separated into a phase having a higher acidic compound content and a phase having a lower acidic compound content due to absorption of the acidic compound. Here, the solubility parameter is represented by the following Formula (3).

$$[ Formula 3 ]$$

$$δ = \sqrt{( Δ H - RT ) / V} \qquad ( 3 )$$

ΔH is a molar evaporation latent heat, R is a gas constant, T is an absolute temperature, and V is a molar volume.

TABLE 1

| | | | Organic solvent/ | | |
|---|---|---|---|---|---|
| Difference between | | | Solubility parameters[(cal/cm³)^{1/2}] | | |
| solubility parameters/ | | | 1-butanol | 1-pentanol | DEGDEE |
| Two-phase separation ability | | | 11.3 | 11.0 | 8.2 |
| Amine | MEA | 14.3 | 3.0 | 3.3 | 6.1 |
| compound/ | | | Good | Good | Not miscible |
| Solubility | AEE | 12.7 | 1.4 | 1.7 | 4.5 |
| parameters | | | Good | Good | Not miscible |
| [(cal/cm³)^{1/2}] | MAE | 12.5 | 1.2 | 1.5 | 4.4 |
| | | | Good | Good | Not miscible |
| | EAE | 12.0 | 0.7 | | 3.8 |
| | | | Not | | Good |
| | | | separated | | |

As shown in Table 1, in the absorbent containing water, the amine compound, and the organic solvent, the combination of the amine compound and the organic solvent is selected such that a value obtained by subtracting the solubility parameter of the organic solvent from the solubility parameter of the amine compound is 1.1 (cal/cm³)^{1/2} or more and 4.2 (cal/cm³)^{1/2} or less. In this case, the absorbent is two-phase separated into a phase having a higher acidic compound content and a phase having a lower acidic compound content by absorption of the acidic compound. In a case where a value of the difference between the solubility parameters is less than the above-described lower limit value, the treatment liquid may not be separated into two phases even if the treatment liquid absorbs the acidic compound. In a case where a value of the difference between the solubility parameters exceeds the above-described upper limit value, the treatment liquid may be separated into two phases before the treatment liquid absorbs the acidic compound. In this case, in a step of bringing the treatment liquid in contact with the gas to be treated containing the acidic compound, the contact state between the treatment liquid and the gas to be treated becomes uneven, and absorption efficiency may be reduced. Note that “Good” in Table 1 means that the treatment liquid was a single liquid phase before absorption of carbon dioxide and was separated into two liquid phases by absorption of carbon dioxide. “Not miscible” in Table 1 means that the treatment liquid was in a two-liquid phase state before absorption of carbon dioxide, and a single liquid phase was not formed. “Not separated” in Table 1 means that the treatment liquid was still a single liquid phase after absorption of carbon dioxide.

It is desirable that the absorption condition in the absorber 21 is set to a region where a larger amount of carbon dioxide is dissolved while the treatment liquid is separated into two phases, and the regeneration condition in the regenerator 22 is set to a region where the treatment liquid is not two-phase separated and carbon dioxide is not dissolved so much. Since the degree of absorption of carbon dioxide changes depending on the temperature, and the ease of phase separation changes, the equilibrium of the carbon dioxide absorption concentration deviates. By utilizing the above, the gas treatment device 10 can suppress a temperature difference between a regeneration temperature and an absorption temperature to be low. In addition, since the separation promoting gas is supplied to the regenerator 22, the regeneration temperature can be suppressed to be lower.

Here, a gas treatment method using the gas treatment device 10 according to the first embodiment will be described. The gas treatment method includes an absorption step, a regeneration step, and a reaction step.

The absorption step is a step of bringing the gas to be treated in contact with the treatment liquid in the absorber 21. The gas to be treated containing at least carbon dioxide is supplied to the absorber 21 through the introduction path 31. The treatment liquid is introduced into the absorber 21 through the second flow path 24b of the circulation path 24. The gas to be treated may have, for example, a temperature and a pressure of 40° C. and 30 kPaG, respectively. The treatment liquid comes in contact with carbon dioxide contained in the gas to be treated and absorbs the carbon dioxide. The gas to be treated that has released carbon dioxide is, for example, 50° C. and 10 kPaG. In the absorber 21, the treatment liquid that has absorbed carbon dioxide is stored. In a case where a treatment liquid that is phase-separated is used, the treatment liquid in contact with carbon dioxide is phase-separated into a first phase portion having a higher content of carbon dioxide and a second phase portion having a lower content of carbon dioxide.

The treatment liquid stored in the absorber 21 is sent to the regenerator 22. At this time, the treatment liquid flowing through the first flow path 24a is heated by the treatment liquid flowing through the second flow path 24b in the heat exchanger 26, and is introduced into the regenerator 22.

The regeneration step is a step of heating the treatment liquid introduced into the regenerator 22 to separate carbon dioxide from the treatment liquid. The treatment liquid in the regenerator 22 is heated to about 60° C. to 90° C. The separation promoting gas flowing into the regenerator 22 through the introduction path 52 comes in contact with the treatment liquid. In the regenerator 22, since the treatment liquid is heated with the separation promoting gas interposed therebetween, the regeneration temperature of carbon dioxide can be suppressed to be lower.

When the treatment liquid is heated in the regenerator 22, water vapor evaporated from the treatment liquid may be obtained. The carbon dioxide and water vapor separated from the treatment liquid flow through the supply path 40. The gas flowing out of the regenerator 22 to the supply path 40 has, for example, a temperature of 50° C. and a pressure of 850 kPaG or less. In the supply path 40, the water vapor is condensed in the condenser 44 and returned to the regenerator 22.

The treatment liquid stored in the regenerator 22 flows through the second flow path 24b and returns to the absorber 21. The treatment liquid has, for example, a temperature of 65° C. and a pressure of 850 kPaG or less. Since the treatment liquid heats the treatment liquid flowing through the first flow path 24a in the heat exchanger 26, the temperature of the treatment liquid decreases.

The gas containing carbon dioxide flowing through the supply path 40 is heated in the preheater 50 by the separation promoting gas flowing through the introduction path 52. That is, the gas flowing through the supply path 40 exchanges heat with the separation promoting gas heated in the reactor 14 in the preheater 50, and is heated. The gas heated by the preheater 50 has, for example, a temperature of 250° C. to 500° C. and a pressure of 850 kPaG or less. The heated (preheated) gas containing carbon dioxide flows into the reactor 14.

The reaction step is performed in the reactor 14. In the reaction step, a synthesis reaction of methane occurs by using the carbon dioxide and the hydrogen introduced through the supply path 40 and the hydrogen introduced through the branch path 52d of the introduction path 52. That is, a part of the hydrogen flowing through the introduction path 52 is used in the reaction step. Since the gas containing carbon dioxide is introduced into the reactor 14 after being preheated by the preheater 50, a methanation reaction can be promoted. The temperature in the reactor 14 is raised by this methanation reaction. Therefore, the separation promoting gas flowing through the heat transfer path 52b of the introduction path 52 is heated by using the heat in the reactor 14 as a heat source. The separation promoting gas (hydrogen gas) introduced into the reactor 14 through the branch path 52d of the introduction path 52 has, for example, a temperature of 25° C. and a pressure of 850 kPaG or less. The separation promoting gas that has flowed through the introduction path 52 and passed through the reactor 14 has a temperature of about 400° C. and a pressure of 850 kPaG or less. On the other hand, a methane gas generated in the reactor 14 has a temperature of 250° C. to 500° C. and a pressure of 850 kPaG or less. The methane gas is led out from the reactor 14 through a lead-out path 55 connected to an upper part of the reactor 14 and sent to a demander of the methane gas.

The separation promoting gas (hydrogen gas) flowing through the heat transfer path 52b of the introduction path 52 is heated by receiving the reaction heat generated in the reactor 14. The heated separation promoting gas flows through the downstream path 52c of the introduction path 52, and is introduced into the regenerator 22 after heating the gas containing carbon dioxide and hydrogen in the preheater 50. The separation promoting gas introduced into the regenerator 22 has, for example, a temperature of 60° C. and a pressure of 850 kPaG or less. That is, the temperature of the separation promoting gas introduced into the regenerator 22 is about the same as the regeneration temperature of the treatment liquid. Therefore, in the regenerator 22, it is possible to suppress using the heat supplied from the reboiler 46 for heating the separation promoting gas.

As described above, in the present embodiment, since the separation promoting gas is introduced into the regenerator 22 through the introduction path 52, the acidic compound can be easily separated from the treatment liquid. Therefore, a lower temperature can be set as the regeneration temperature in the regenerator 22, and the acidic compound can be separated with lower energy. In addition, before the separation promoting gas is introduced into the regenerator 22, the separation promoting gas is preheated by using heat generated in the reactor 14 as a heat source. It is therefore possible to suppress consuming the heat to be used for regenerating the treatment liquid in the regenerator 22 for heating the separation promoting gas. As a result, it is possible to suppress a decrease in the separation efficiency of the acidic compound from the treatment liquid. Since the heat generated in the reactor 14 is effectively used, it is not necessary to separately prepare a heat source for preheating the separation promoting gas.

The gas containing carbon dioxide generated in the regenerator 22 is heated in the preheater 50 before being introduced into the reactor 14. Therefore, even when the regeneration temperature of the treatment liquid in the regeneration step in the regenerator 22 is low, the gas containing carbon dioxide can be efficiently used for the reaction in the reactor 14. Moreover, since the heat generated in the reactor 14 is used in the regenerator 22 via the separation promoting gas, the heat balance can be further enhanced.

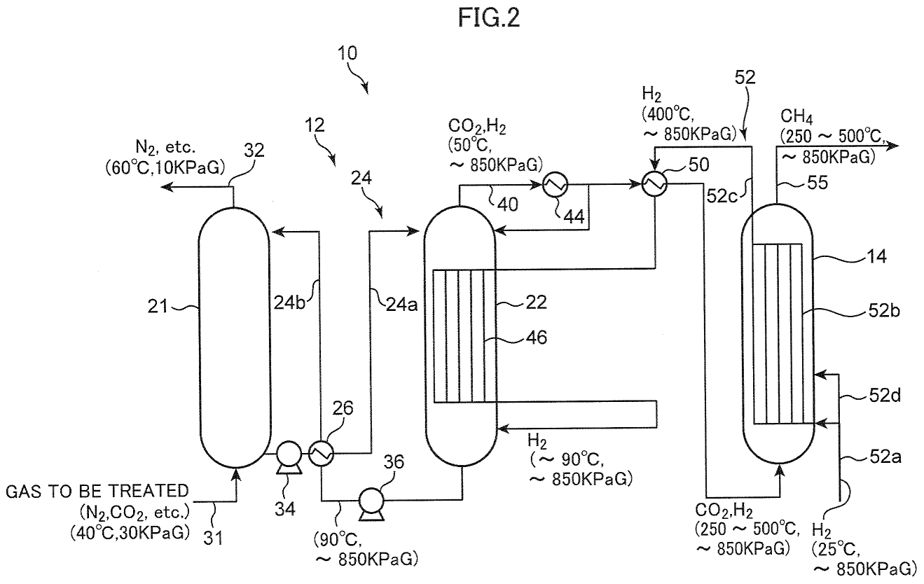

In the present embodiment, the reboiler 46 is disposed outside the regenerator 22 and is configured to directly or indirectly heat the treatment liquid by an arbitrary heat source such as electricity, steam, or a burner. However, the present embodiment is not limited to this configuration. For example, as illustrated in FIG. 2, the reboiler 46 may be connected to the downstream path 52c of the introduction path 52 and disposed in the regenerator 22 (or in contact with the regenerator 22). In this case, since the separation promoting gas heated in the reactor 14 flows in the reboiler 46, the heat generated in the reactor 14 can be used as the heat source of the reboiler 46.

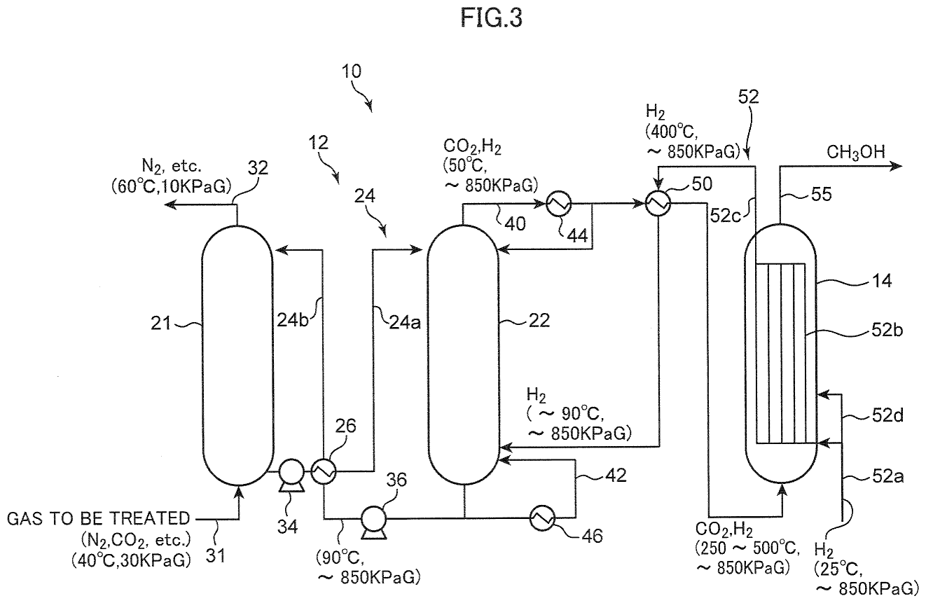

In the present embodiment, the reactor 14 is configured as a reactor for synthesizing methane, but instead, as illustrated in FIG. 3, the reactor 14 may be configured as a reactor for synthesizing methanol or ethanol. In this case, the reactor 14 may be configured to proceed a hydrogenation reaction of carbon dioxide in the presence of a catalyst to synthesize methanol or ethanol, or may be configured to react hydrogen with carbon monoxide in the presence of a catalyst to synthesize methanol or ethanol. When carbon monoxide is used, carbon monoxide may be generated by a reverse shift reaction as in the reaction formula (1).

Although the preheater 50 is provided in the present embodiment, the preheater 50 may be omitted.

### Second Embodiment

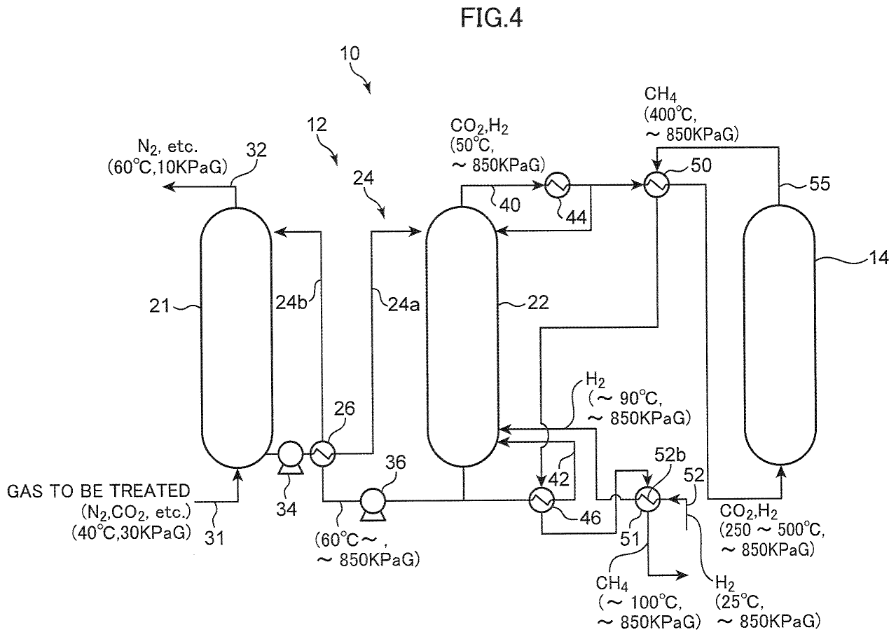

In the first embodiment, the introduction path 52 for introducing the separation promoting gas into the regenerator 22 passes through the reactor 14, and the heat transfer path 52b for preheating the separation promoting gas is provided in the reactor 14. On the other hand, in a second embodiment, as illustrated in FIG. 4, the introduction path 52 does not pass through the reactor 14, and the heat transfer path 52b is provided outside the reactor 14. Specifically, the heat transfer path 52b is provided in the lead-out path 55 for leading out from the reactor 14 a high-temperature gas (methane gas) generated by the methanation reaction in the reactor 14, and receives the heat of the reactor 14 via the lead-out path 55. The heat transfer path 52b is provided in the preheater 51 that exchanges heat between the high-temperature gas from the reactor 14 and the separation promoting gas flowing through the introduction path 52.

The reaction for generating methane in the reactor 14 is an exothermic reaction. In order to stably operate the reactor 14, it is necessary to cause a reaction while adjusting the temperature of the reactor 14 to a constant temperature. Therefore, in the configuration in which the separation promoting gas is introduced into the reactor 14, it may be difficult to adjust the temperature of the reactor 14 by the separation promoting gas. Therefore, in the present embodiment, the heat transfer path 52b for preheating the separation promoting gas is disposed at a place different from the reactor 14. The temperature of the reactor 14 may be adjusted by using water, oil, or the like, but since the temperature of the gas after the reaction flowing out of the reactor 14 is about 400° C., the heat generated in the reactor 14 is preferably used effectively.

In the second embodiment, as illustrated in FIG. 4, the lead-out path 55 for leading out the gas after the reaction (methane gas) from the reactor 14 is connected to the upper part of the reactor 14. The lead-out path 55 is provided with the preheater 50, the reboiler 46, and the heat transfer path 52b (preheater 51). The temperature of the reactor 14 is adjusted to, for example, about 400° C. in order to promote the methanation reaction. Therefore, the methane gas generated in the reactor 14 and led out from the reactor 14 through the lead-out path 55 has, for example, a temperature of about 400° C. and a pressure of 850 kPaG or less. The methane gas generated in the reactor 14 first heats the gas flowing through the supply path 40 (the gas containing the acidic compound obtained in the regenerator 22) in the preheater 50, then heats the treatment liquid in the reboiler 46, and further heats the separation promoting gas in the heat transfer path 52b. Therefore, the temperature of the methane gas flowing through the lead-out path 55 decreases every time the methane gas passes through the preheater 50, the reboiler 46, and the heat transfer path 52b (preheater 51), and becomes lower than 100° C. when the methane gas flows out from the heat transfer path 52b (preheater 51). In the heat transfer path 52b, the separation promoting gas such as hydrogen gas having a temperature of about 25° C. (normal temperature) is heated to a temperature of less than about 90° C. The heated separation promoting gas is introduced into the regenerator 22. The preheating of the hydrogen gas and the carbon dioxide gas flowing through the supply path 40, the heating in the reboiler 46 for the endothermic reaction required in the regenerator 22, and the preheating of the hydrogen gas in the introduction path 52 are performed by using the heat of the exothermic reaction in the reactor 14 as a heat source. Therefore, heat can be effectively used. The supply source of hydrogen gas may be constituted by, for example, a water electrolyzer configured to be able to produce hydrogen by using renewable energy.

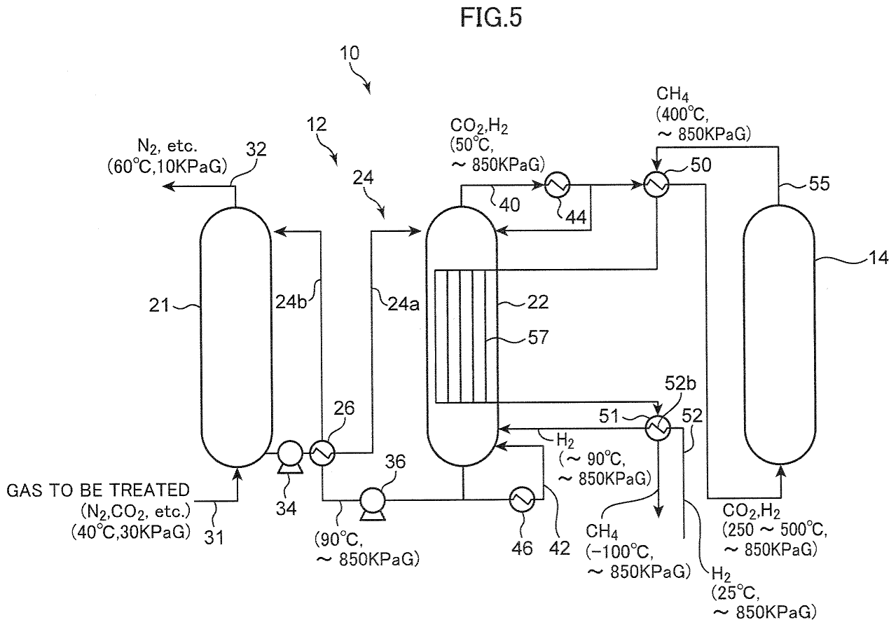

In the configuration illustrated in FIG. 4, the lead-out path 55 also passes through the reboiler 46 in the heating flow path 42. However, in the configuration illustrated in FIG. 5, the reboiler 46 of the heating flow path 42 is configured to heat the treatment liquid by an arbitrary heat source instead of the heat of the gas from the reactor 14. On the other hand, the lead-out path 55 is also connected to the heat exchanger 57 provided in the regenerator 22. The heat exchanger 57 functions as a reboiler (second reboiler) that heats the treatment liquid in the regenerator 22. The heat exchanger 57 is disposed upstream of the heat transfer path 52b (preheater 51) in the lead-out path 55. The methane gas that has passed through the preheater 50 heats the treatment liquid in the heat exchanger 57 which is the second reboiler, and then heats the separation promoting gas in the heat transfer path 52b (preheater 51).

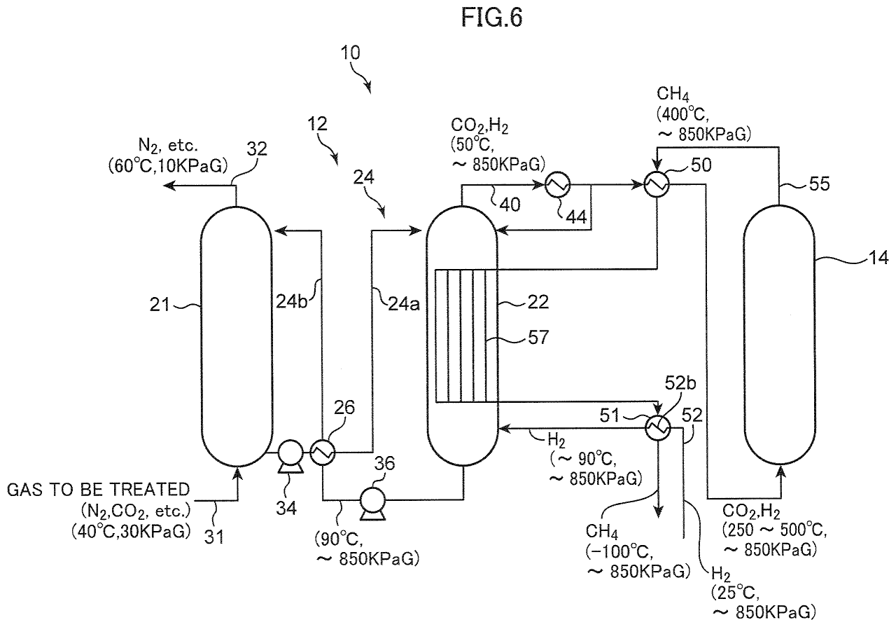

In the configuration in FIG. 5, the reboiler 46 is provided, but since the heat exchanger 57 functioning as a reboiler is disposed in the regenerator 22, the heating flow path 42 and the reboiler 46 can be omitted as illustrated in FIG. 6. When the heat exchanger 57 functioning as a reboiler is disposed in the regenerator 22, the first flow path 24a is preferably connected to a portion of the regenerator 22 above the heat exchanger 57. The second flow path 24b is preferably connected to a portion of the regenerator 22 below the heat exchanger 57.

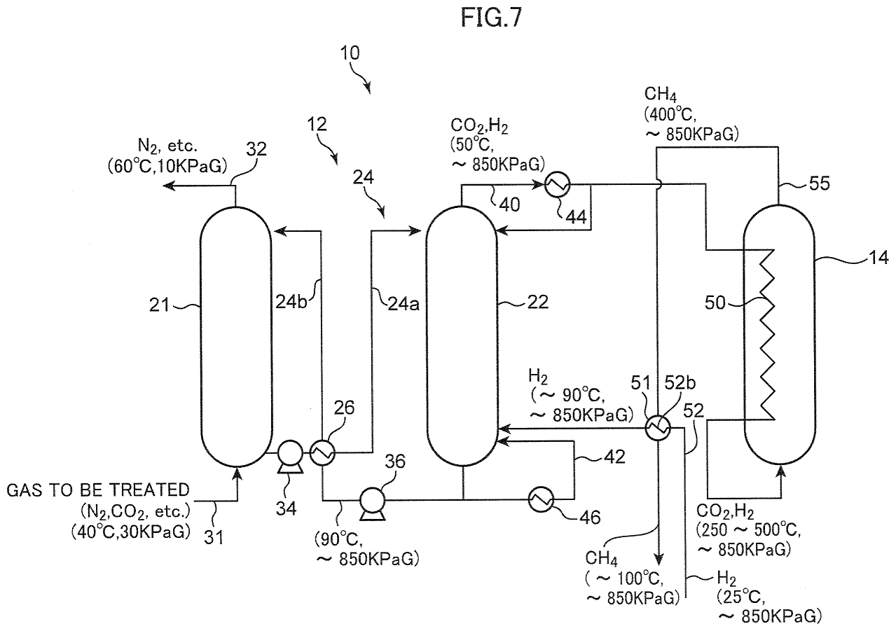

As illustrated in FIG. 7, the preheater 50 may be disposed in the reactor 14. That is, the supply path 40 passes through the inside of the reactor 14 (or is disposed to be in contact with the reactor 14), and the preheater 50 may be provided in a portion passing through the inside of the reactor 14 (or a portion in contact with the reactor 14). The gas containing hydrogen and carbon dioxide in the preheater 50 heated by the gas after the reaction in the reactor 14 flows out of the preheater 50 through the supply path 40, is then introduced into the reactor 14, and is subjected to the methanation reaction. The high-temperature methane gas generated by the methanation reaction is sent to the heat transfer path 52b through the lead-out path 55 to heat a separation promoting gas.

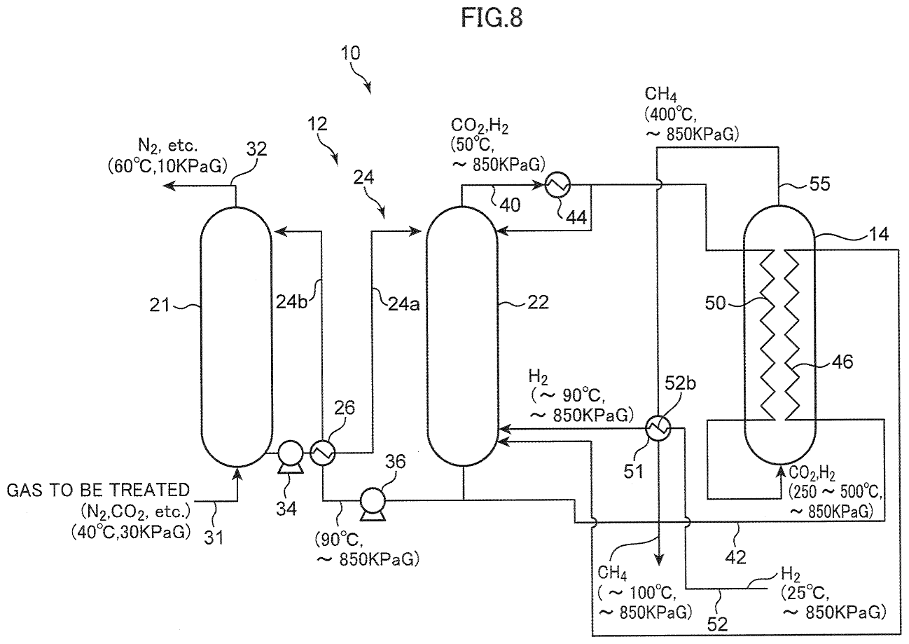

In the configuration in FIG. 7, the reboiler 46 is disposed separately from the regenerator 22 and the reactor 14. Alternatively, in the configuration in FIG. 8, the reboiler 46 is disposed in the reactor 14 (or disposed in contact with the reactor 14). That is, the reboiler 46 may use heat generated by the exothermic reaction in the reactor 14 as a heat source. In this case, the preheater 50 may be at the position in FIG. 6 instead of the position in FIG. 8.

It should be understood that the embodiments disclosed herein are illustrative in all respects and are not restrictive. The present invention is not limited to the above embodiments, and various changes, improvements, and the like can be made without departing from the gist of the present invention.

Here, the above embodiments will be outlined.
(1) A gas treatment method according to the embodiment includes an absorption step of bringing a gas to be treated containing an acidic compound that generates an acid when dissolved in water in contact with a treatment liquid in an absorber to cause the acidic compound contained in the gas to be treated to be absorbed by the treatment liquid; a regeneration step of heating the treatment liquid that has absorbed the acidic compound in a regenerator to separate the acidic compound from the treatment liquid; and a reaction step of causing a synthesis reaction by using the gas containing the acidic compound separated from the treatment liquid in the regeneration step. In the regeneration step, a separation promoting gas that promotes separation of the acidic compound from the treatment liquid is introduced into the regenerator, and the separation promoting gas is preheated by using heat generated in the reaction step as a heat source before being introduced into the regenerator.

In the gas treatment method, since the separation promoting gas is used in the regeneration step, the acidic compound can be easily separated from the treatment liquid. Therefore, a lower temperature can be set as the regeneration temperature in the regenerator, and the acidic compound can be separated with lower energy. In addition, before the separation promoting gas is introduced into the regenerator, the separation promoting gas is preheated by using heat generated in the reaction step as a heat source. It is therefore possible to suppress the heat to be used for regenerating the treatment liquid in the regeneration step consumed for heating the separation promoting gas. As a result, it is possible to suppress a decrease in the separation efficiency of the acidic compound from the treatment liquid. Since the heat generated in the reaction step is effectively used, it is not necessary to separately prepare a heat source for preheating the separation promoting gas.
(2) The treatment liquid may include a treatment liquid that is phase-separated by absorption of the acidic compound. In this case, the separation promoting gas may include a gas that is hardly dissolved in the treatment liquid, water vapor, or a mixed gas of a water vapor and a gas that is hardly dissolved in the treatment liquid. In this aspect, in the regeneration step, not only the separation promoting gas that is a gas that is hardly dissolved in the treatment liquid is brought into contact with the treatment liquid, but also the acidic compound is separated from the treatment liquid with a phase portion having a low acidic compound content interposed therebetween. Therefore, the energy required for regeneration of the treatment liquid can be further reduced. Even in the case of water vapor or a mixed gas of water vapor and a gas that is hardly dissolved in the treatment liquid, energy required for regeneration can be reduced by bringing the water vapor in contact with the treatment liquid in the regeneration step.
(3) The reaction step may be any one of a step of synthesizing methane, a step of synthesizing methanol, or a step of synthesizing ethanol. In this aspect, methane, methanol, or ethanol is synthesized by using an acidic compound contained in the gas separated from the treatment liquid.
(4) The separation promoting gas may contain hydrogen gas. In this case, a part of the hydrogen gas may be used in the reaction step. In this aspect, a part of the hydrogen gas is used for promoting separation, and the other part is used in the reaction step.
(5) The gas generated in the regeneration step may be preheated by the separation promoting gas preheated in the reaction step before being used in the reaction step.

In this aspect, since the gas generated in the regeneration step is heated before being used in the reaction step, the gas can be efficiently used in the reaction step even when the regeneration temperature in the regeneration step is low. Moreover, since the heat generated in the reaction step is used in the regeneration step via the separation promoting gas, the heat balance can be further enhanced.
(6) A gas treatment device according to the embodiment includes an absorber that brings a gas to be treated containing an acidic compound that generates an acid when dissolved in water in contact with a treatment liquid to cause the acidic compound contained in the gas to be treated to be absorbed by the treatment liquid, a regenerator that heats the treatment liquid that has absorbed the acidic compound to separate the acidic compound from the treatment liquid, a reactor that causes a synthesis reaction by using a gas containing the acidic compound separated from the treatment liquid in the regenerator, and an introduction path that introduces, into the regenerator, a separation promoting gas that promotes separation of the acidic compound from the treatment liquid. The introduction path includes a heat transfer path that preheats the separation promoting gas before introduction into the regenerator by using heat generated in the reactor as a heat source.

In the gas treatment device, since the separation promoting gas is introduced into the regenerator through the introduction path, the acidic compound can be easily separated from the treatment liquid. Therefore, a lower temperature can be set as the regeneration temperature in the regenerator, and the acidic compound can be separated with lower energy. In addition, before the separation promoting gas is introduced into the regenerator, the separation promoting gas is preheated by using heat generated in the reactor as a heat source. It is therefore possible to suppress the heat to be used for regenerating the treatment liquid in the regenerator consumed for heating the separation promoting gas. As a result, it is possible to suppress a decrease in the separation efficiency of the acidic compound from the treatment liquid. Since the heat generated in the reactor is effectively used, it is not necessary to separately prepare a heat source for preheating the separation promoting gas.
(7) The treatment liquid may include a treatment liquid that is phase-separated by absorption of the acidic compound. In this case, the separation promoting gas may include a gas that is hardly dissolved in the treatment liquid, water vapor, or a mixed gas of a water vapor and a gas that is hardly dissolved in the treatment liquid. In this aspect, in the regenerator, not only the separation promoting gas that is a gas that is hardly dissolved in the treatment liquid is brought into contact with the treatment liquid, but also the acidic compound is separated from the treatment liquid with a phase portion having a low acidic compound content interposed therebetween. Therefore, the energy required for regeneration of the treatment liquid can be further reduced. Even in the case of water vapor or a mixed gas of water vapor and a gas that is hardly dissolved in the treatment liquid, energy required for regeneration can be reduced by bringing the water vapor in contact with the treatment liquid in the regenerator.
(8) The reactor may be any one of a reactor that synthesizes methane, a reactor that synthesizes methanol, or a reactor that synthesizes ethanol.
(9) The separation promoting gas may contain hydrogen gas. In this case, the introduction path may include a branch path that introduces a part of the hydrogen gas into the reactor. In this aspect, a part of the hydrogen gas flowing through the introduction path can be used for promoting separation, and the other part can be used in the reactor.
(10) The gas treatment device may further include a preheater that preheats the gas generated in the regenerator and before being introduced into the reactor by using the heat generated in the reactor as a heat source.

In this aspect, since the gas generated in the regenerator is heated before being introduced into the reactor, the gas can be efficiently used in the reactor even when the regeneration temperature of the treatment liquid in the regenerator in the regeneration step is low. Moreover, since the heat generated in the reactor is used in the regenerator, the heat balance can be further enhanced.

As described above, it is possible to effectively reduce energy required for regeneration of the treatment liquid.

This application is based on Japanese Patent Application No. 2023-020773 filed with the Japan Patent Office on Feb. 14, 2023, the contents of which are incorporated herein by reference.

Although the present invention has been appropriately and sufficiently described through the embodiments with reference to the above drawings to express the present invention, it should be recognized that a person skilled in the art can easily modify and/or improve the above-described embodiments. Therefore, unless a change or improvement made by a person skilled in the art is at a level departing from the scope of rights of the claims described in claims, the change or improvement is interpreted to be included in the scope of rights of the claims.

1. A gas treatment method comprising:
an absorption step of bringing a gas to be treated containing an acidic compound that generates an acid when dissolved in water in contact with a treatment liquid in an absorber to cause the acidic compound contained in the gas to be treated to be absorbed by the treatment liquid;
a regeneration step of heating the treatment liquid that has absorbed the acidic compound in a regenerator to separate the acidic compound from the treatment liquid; and
a reaction step of causing a synthesis reaction by using the gas containing the acidic compound separated from the treatment liquid in the regeneration step,
wherein in the regeneration step, a separation promoting gas that promotes separation of the acidic compound from the treatment liquid is introduced into the regenerator, and
the separation promoting gas is preheated by using heat generated in the reaction step as a heat source before being introduced into the regenerator.

2. The gas treatment method according to claim 1, wherein
the treatment liquid includes a treatment liquid that is phase-separated by absorption of the acidic compound, and
the separation promoting gas includes a gas that is hardly dissolved in the treatment liquid, water vapor, or a mixed gas of a water vapor and a gas that is hardly dissolved in the treatment liquid.

3. The gas treatment method according to claim 1, wherein the reaction step is any one of a step of synthesizing methane, a step of synthesizing methanol, or a step of synthesizing ethanol.

4. The gas treatment method according to claim 3, wherein
the separation promoting gas contains hydrogen gas, and
a part of the hydrogen gas is used in the reaction step.

5. The gas treatment method according to claim 1, wherein the gas generated in the regeneration step is preheated by the separation promoting gas preheated in the reaction step before being used in the reaction step.

6. A gas treatment device comprising:
an absorber configured to bring a gas to be treated containing an acidic compound that generates an acid when dissolved in water in contact with a treatment liquid to cause the acidic compound contained in the gas to be treated to be absorbed by the treatment liquid;
a regenerator configured to heat the treatment liquid that has absorbed the acidic compound to separate the acidic compound from the treatment liquid;
a reactor configured to cause a synthesis reaction by using a gas containing the acidic compound separated from the treatment liquid in the regenerator; and
an introduction path configured to introduce, into the regenerator, a separation promoting gas that promotes separation of the acidic compound from the treatment liquid,
wherein the introduction path includes a heat transfer path configured to preheat the separation promoting gas before introduction into the regenerator by using heat generated in the reactor as a heat source.

7. The gas treatment device according to claim 6, wherein
the treatment liquid includes a treatment liquid that is phase-separated by absorption of the acidic compound, and
the separation promoting gas includes a gas that is hardly dissolved in the treatment liquid, water vapor, or a mixed gas of a water vapor and a gas that is hardly dissolved in the treatment liquid.

8. The gas treatment device according to claim 6, wherein the reactor is any one of a reactor configured to synthesize methane, a reactor configured to synthesize methanol, or a reactor configured to synthesize ethanol.

9. The gas treatment device according to claim 8, wherein
the separation promoting gas contains hydrogen gas, and
the introduction path includes a branch path configured to introduce a part of the hydrogen gas into the reactor.

10. The gas treatment device according to claim 6, further comprising a preheater configured to preheat the gas generated in the regenerator and before being introduced into the reactor by using the heat generated in the reactor as a heat source.
